(19) United States
(12) Patent Application Publication
(10) Pub. No.: US 2015/0051296 A1
(43) Pub. Date: Feb. 19, 2015

(54) USE OF FATTY ACID TO REDUCE SKIN IRRITATION ASSOCIATED WITH RETINOID THERAPY

(71) Applicants: John F. Rebhun, Greenville, MI (US); Rodney A. Velliquette, Ada, MI (US); Samantha Westveer, Kentwood, MI (US)

(72) Inventors: John F. Rebhun, Greenville, MI (US); Rodney A. Velliquette, Ada, MI (US); Samantha Westveer, Kentwood, MI (US)

(73) Assignee: Access Business Group Internaional LLC, Ada, MI (US)

(21) Appl. No.: 14/454,146

(22) Filed: Aug. 7, 2014

Related U.S. Application Data
(60) Provisional application No. 61/865,425, filed on Aug. 13, 2013.

(51) Int. Cl. A61K 8/36; A61Q 19/08; A61Q 19/00; A61K 8/34

(52) U.S. Cl. CPC A61K 8/361; A61K 8/342; A61Q 19/08; A61Q 19/00; A61K 2800/75
USPC 514/725

(57) ABSTRACT

A composition and a method that is effective to reduce, limit, or diminish one or more side effects of a retinoid are described. The composition includes a retinoid and a free fatty acid. The composition may further include one or more cosmetically suitable ingredients so long as they do not interfere with the efficaciousness of the free fatty acid. The method for reducing, limiting, or diminishing a side effect associated with the topical administration of a retinoid to a skin area of a subject includes topically administering a free fatty acid to the skin area prior to, during, and/or after topical administration of the retinoid.

### RELATED APPLICATIONS

The present patent document claims the benefit of the filing date under 35 U.S.C. §119(e) of Provisional U.S. Patent Application Ser. No. 61/865,425, filed Aug. 13, 2013, which is hereby incorporated by reference.

### BACKGROUND

The present disclosure relates generally to cosmetics and, more particularly, to the use of free fatty acids to reduce skin irritation associated with retinoid therapy.

Retinoids are a class of compounds known to be useful for treating disorders of the skin. Numerous compounds defined as retinoids are known, and several are currently used to treat a variety of disorders. Such disorders may include acne (e.g., acne vulgaris, cystic acne, and acne rosacea), warts, eczema (e.g., hyperkaratotic eczema of the hands and feet), psoriasis, aged skin (e.g., photoaged skin or chronologically aged skin), skin cancer, post-inflammatory hyperpigmentation in black skin, cutaneous lesions of AIDS-related Kaposi's sarcoma, keratinization disorders (e.g., ichthyoses, pityriasis rubra pilaris, and Darier's disease), carcinoma (e.g., basal cell and squamous cell carcinomas), keratocanthoma, and butaneous sarcoidosis. For example, 13-cis-retinoic acid (available as Accutane®, Roche Pharmaceuticals, Basel, Switzerland) is used to treat skin disorders such as severe recalcitrant nodular acne, and retinoic acid (available as Retin-A®, Ortho Dermatologics, Los Angeles, Calif.) is used to treat acne vulgaris. Retinoids also may be administered to prevent conditions such as photoaging of skin, chronological aging of skin, UV-induced loss of collagen biosynthesis, UV-induced functional vitamin A deficiency, and scarring and inflammation due to acne.

Retinoids are also efficacious in treating dermal related disorders such as wrinkles, aging related skin changes, abnormal or diminished synthesis of collagen and elastin, diminished levels of collagen and elastin in the dermis, stretch marks, skin lines, fine lines, thinning of skin, skin thickening due to elastosis of photoaging, loss or reduction of skin resiliency, elasticity and recoilability, lack of skin resiliency, elasticity and recoilability, and older looking skin.

Retinoids can be administered both topically and systemically. Topical administration of retinoids, however, is associated with undesirable side effects such as skin irritation, dryness, redness, and epidermal hyperplasia. For example, in keratinocytes, retinoids have been known to induce proliferation, causing epidermal hyperplasia, edema, blistering, crusting, severe local erythema, burning, stinging, pruritus, heightened burning susceptibility upon exposure to sunlight, skin peeling, and overall physical discomfort.

Although retinoids are clinically useful and widely prescribed, it would be desirable to eliminate or at least minimize the undesirable side effects associated with administration of retinoids without diminishing the beneficial effects of the retinoids.

### SUMMARY

The present invention includes a composition that is effective to reduce, limit, or diminish one or more side effects of a retinoid wherein the composition includes a retinoid and a free fatty acid. The composition may further include one or more cosmetically suitable ingredients so long as they do not interfere with the efficaciousness of the free fatty acid.

The present invention also includes a method for reducing, limiting, or diminishing a side effect associated with the topical administration of a retinoid to a skin area of a subject wherein the method includes topically administering a free fatty acid to the skin area prior to, during, and/or after topical administration of the retinoid.

These and other features and advantages of the invention will become apparent upon consideration of the following detailed description of the presently preferred embodiments, viewed in conjunction with the appended drawings.

### BRIEF DESCRIPTION OF THE DRAWINGS

The patent or application file contains at least one drawing (color photographs) executed in color. Copies of this patent or patent application publication with color drawing(s) will be provided by the Office upon request and payment of the necessary fee.

FIG. 1 is a color photograph that compares the skin irritation associated with administration of (A) a lotion including a retinoid without a fatty acid, (B) a lotion composition including a retinoid and a fatty acid according to at least one embodiment of the present invention, and (C) a base lotion including a fatty acid without a retinoid.

FIG. 2 is a color photograph that compares skin irritation associated with administration of (A) a lotion including a retinoid without a fatty acid, (B) a base lotion including a fatty acid without a retinoid, (C) one exemplary lotion composition including a retinoid and a fatty acid according to the present invention, and (D) another exemplary lotion composition including a retinoid and a fatty acid according to the present invention.

FIG. 3 is a graph of the response of human kidney cells stably transfected with the retinoic acid response element (RARE) construct to increasing amounts of retinoic acid in the presence of increasing amounts of oleic acid.

FIG. 4 shows a chemical structure of Borage Oil triglyceride with palmitic, linoleic and oleic acid (18:1) as side chains.

FIG. 5 is a graph showing activation of Peroxisome Proliferator Activated Receptor by free fatty acid (i.e., oleic acid 18:1) and not by Borage Oil.

### DETAILED DESCRIPTION

As used herein, the term “effective amount” or “therapeutically effective amount” refers to an amount effective at dosages and for periods of time sufficient to achieve a desired result.

As used herein, the term “subject” refers to any animal (e.g., a mammal) including, but not limited to, humans, non-human primates, rodents, and the like, to which a compound may be administered.

As used herein, the term “carrier” refers to a composition that aids in maintaining one or more components (e.g., effective ingredients) of a composition in a soluble and homogeneous state in a form suitable for administration, which is nontoxic and which does not interact with other components in a deleterious manner.

As used herein, the term “retinoid” refers to any of a class of compounds chemically related to vitamin A. They may be natural or synthetic and include analogs. The retinoid may include a β-ionone ring and a polyunsaturated side chain. The polyunsaturated side chain may include an alcohol, an aldehyde, a carboxylic acid group, or an ester group. Examples of retinoids may include, but are not limited to, retinol (vitamin A), retinal (i.e., retinaldehyde), retinoic acid (vitamin A acid), tretinoin (i.e., retinoic acid), isotretinoin, alitretinoin, etretinate, acitretin, tazarotene, bexarotene, adapalene, any other conventional retinoids, derivatives thereof, and combinations thereof. For example, a retinoid may include all-trans retinal, all-trans retinol, all-trans retinoic acid, 9-cis-retinoic acid, 13-cis-retinal, 13-cis-retinol, 9-cis-retinal, 13-cis-retinoic acid, 9-cis-retinal, or combinations thereof.

As used herein, the term “fatty acid” refers to a saturated or unsaturated monocarboxylic acid having an aliphatic tail, which may include from about 4 to about 28 carbon atoms. The fatty acid may be a saturated monocarboxylic acid having the general formula CₙH₂ₙ₊₁COOH, wherein n is a positive integer. In one example, n may be from about 4 to about 28. The aliphatic tail of the fatty acid may be free of hydroxyl functional groups. The fatty acid may occur naturally in the form of esters in fats, waxes, and essential oils or in the form of glycerides in fats and fatty oils. Examples of fatty acids may include, but are not limited to, oleic acid, myristic, palmitic, rumenic, vaccenic, myrisoleic, palmitoleic, alpha-linoleic acid. It may also include any other conventional fatty acids, derivatives thereof, and combinations thereof. In some embodiments, a suitable fatty acid does not include arachidonic acid.

As used herein, the term “free fatty acid” refers to a fatty acid that is unbound to other molecules or constituents. In other words, for purposes of this application, a fatty acid present in oil would not be considered to be a free fatty acid. In this regard, U.S. Pat. No. 5,989,572 describes borage seed oil which is stated to contain gamma-linoleic acid, sterols, tocopherols, linoleic acid, oleic acid, palmitic, amabiline etc. For purposes of this application, the gamma-linoleic acid, linoleic acid, oleic acid, palmitic acid, or any other fatty acid present in the borage seed oil are not considered to be a free fatty acid because they are bound with and are a constituent of the oil—thus, they are not free.

Unless indicated otherwise, all proportions and percentages recited throughout this disclosure are by weight.

Retinoid therapy may include administration of a retinoid to a subject. The retinoid may be administered by any suitable route. For example, the retinoid may be administered topically. The retinoid may be administered to treat or prevent any condition or disorder of the skin. For example, the retinoid may be administered to treat or prevent one or more of acne (e.g., acne vulgaris, cystic acne, and acne rosacea), warts, eczema (e.g., hyperkaratotic eczema of the hands and feet), psoriasis, aged skin (e.g., photoaged skin or chronologically aged skin), UV-induced loss of collagen biosynthesis, UV-induced functional vitamin A deficiency, scarring and/or inflammation due to acne, skin cancer, post-inflammatory hyperpigmentation in black skin, cutaneous lesions of AIDS-related Kaposi's sarcoma, keratinization disorders (e.g., ichthyoses, pityriasis rubra pilaris, and Darier's disease), carcinoma (e.g., basal cell and squamous cell carcinomas), keratocanthoma, and butaneous sarcoidosis. The retinoid may be administered to treat or reduce wrinkles, aging related skin changes, abnormal or diminished synthesis of collagen and elastin, diminished levels of collagen and elastin in the dermis, stretch marks, skin lines, fine lines, thinning of skin, skin thickening due to elastosis of photoaging, loss or reduction of skin resiliency, elasticity and recoilability, lack of skin resiliency, elasticity and recoilability, and older looking skin. The retinoid may include any known retinoid as described herein. In one example, the retinoid may include retinol or a derivative thereof.

A fatty acid may be administered to diminish or limit a side effect associated with retinoid therapy. The fatty acid may be administered in conjunction with retinoid therapy (e.g., prior to, during, and/or after administration of a retinoid). The fatty acid may be administered by any suitable route. For example, the fatty acid may be administered topically. For example, the fatty acid may be administered to diminish or limit one or more of skin irritation, dryness, redness, epidermal hyperplasia, edema, blistering, crusting, erythema, burning, stinging, pruritus, heightened burning susceptibility upon exposure to sunlight, skin peeling, and overall physical discomfort. The fatty acid may include any known fatty acid as described herein. In one example, the fatty acid may include oleic acid (i.e., oleic acid, 18:1) or a derivative thereof.

The fatty acid may be present as a free fatty acid. By providing a free fatty acid, the fatty acid is readily available to act on cellular receptors to activate specific cellular pathways. Administration of a bound fatty acid requires conversion to a free form before beneficial effects would be seen. Oils, as defined, do not contain free fatty acids and do not activate cellular receptors.

Administration of the free fatty acid may aid in rendering the retinoid therapy more tolerable for the subject. This, in turn, may increase the likelihood that the subject will continue the retinoid therapy to achieve the retinoid's beneficial effects. The free fatty acid may be capable of activating peroxisome proliferator activated receptor alpha (PPAR-α). Activation of PPAR-α may promote uptake, utilization, and catabolism of fatty acids by upregulation of genes involved in fatty acid transport, fatty binding and activation, and peroxisomal and mitochondrial fatty acid β-oxidation. Administration of the fatty acid may decrease accelerated barrier disruption, as measured by transepidermal water loss (TEWL), and/or irritation caused by retinoid therapy as further described below.

In one example, a composition may include a retinoid and a fatty acid. The fatty acid may be present as a free fatty acid. The retinoid and the fatty acid may be present together in the composition. Accordingly, the retinoid and the fatty acid may be administered concurrently upon administration of the composition to a subject. The composition may be provided in a form suitable for topical administration to the subject. For example, the composition may be provided in a form selected from the group consisting of an aerosol spray, a gel, a cream, a dispersion, an emulsion, a foam, a liquid, a lotion, a mousse, a patch, a pomade, a powder, a pump spray, a solid, a solution, a stick, a towelette, a salve, and combinations thereof. Suitable emulsions may include, for example, oil-in-water, water-in-oil, and water-in-silicone emulsions. The composition may be formulated in an acceptable carrier. The composition may be prepared, packaged, and labeled for treating any skin disorder including those described herein.

In one example, the composition may include an effective amount of the retinoid and an effective amount of the fatty acid. The composition may include a cosmetically acceptable vehicle. The composition may have a pH from about 6 to about 8 and may have a pH from 6 to 8. For example, the composition may have a pH that is substantially neutral, i.e., a pH of 7.

The composition may include a cosmetically acceptable vehicle or carrier. Examples of cosmetically acceptable vehicles may include, but are not limited to, water, glycerin, various alcohols (e.g., ethanol and propyl alcohol), vegetable oil, mineral oil, silicone oils, fatty ethers, fatty esters, fatty alcohols, glycols, polyglycols, and combinations thereof. Methods of formulation are well known in the art and are disclosed, for example, in Remington's Pharmaceutical Sciences, Gennaro, Mack Publishing Co., Easton Pa., 1990, which is incorporated herein by reference.

The composition may include additional ingredients that do not interfere with the retinoid therapy or the action of the fatty acid. Preferred additional ingredients may include those that would help to further alleviate the side effects of the retinoid therapy. Such additional ingredients may include, for example, humectants, moisturizers, emollients, mild antibacterials (e.g., benzalkonium chloride), mild anesthetics (e.g., benzocaine), and combinations thereof. The additional ingredients may include any other conventional ingredients including, for example, fragrances, colorants, and the like.

In one example, the retinoid may be present in an amount of about 0.01% to about 5%, or about 0.02% to about 2.5%, or about 0.05% to about 1%, or about 0.1% to about 0.5%, or about 0.4% by weight of the composition. In other embodiments, the retinoid may be present in an amount from about 0.02% to about 0.2% by weight of the composition. In one example, the fatty acid may be present in an amount of about 0.01% to about 5%, or about 0.02% to about 2.5%, or about 0.05% to about 1%, or about 0.25% to about 1% by weight of the composition. The retinoid and the fatty acid may be present in the composition in a weight ratio of about 0.4:1 to about 1.6:10.05:1.

The effective dosage of active ingredients employed may vary depending on the particular compound employed, the mode of administration, and the severity of the condition being treated. The composition may be administered as needed, daily, several times per day, or in any suitable regimen such that the desired outcome is achieved. A suitable regimen may include application to the skin twice daily, with one application in the morning and one in the evening. Other suitable regimens may include application to the skin from four times daily to once weekly, although more or less frequent application may be desirable depending on the condition being treated and the subject's response.

### EXAMPLES

### Example 1

A lotion was topically administered to a skin area of a subject and the lotion included 0.4% retinol and the following cosmetic ingredients: water phase containing glycerin (3%), and sorbitan stearate (1%); and oil phase containing isostearyl palmitate (3%), glyceryl trioctanoate (2%), glyceryl stearate (5%), cetyl alcohol, shea butter (1%), dimethicone (1%), and tocopheryl acetate (0.1%). The lotion was substantially free of any fatty acid. To administer the lotion, 0.1 mL of the lotion was applied to a 0.75 in square bandage, and the bandage was applied to a skin area on the subject's forearm. The lotion was administered twice per day for five days, at which time erythema was observed at the treated skin area.

The amount of retinol remaining in the lotion at the end of administration was also measured using high performance liquid chromatography (HPLC) analysis. The amount of retinol remaining in the lotion at the end of administration was 0.34%.

### Example 2

Oleic acid, 18:1, was added to the lotion used in Example 1 to form a lotion composition that included 0.4% retinol and 0.4% oleic acid. The lotion composition was topically administered to a skin area of a subject. To administer the lotion composition, 0.1 mL of the lotion composition was applied to a 0.75 in square bandage, and the bandage was applied to a skin area on the subject's forearm. The lotion composition was administered twice per day for five days, at which time erythema was observed at the treated skin area.

The amount of retinol remaining in the lotion composition at the end of administration was measured using HPLC analysis. The amount of retinol remaining in the lotion composition at the end of administration was 0.33%.

### Example 3

A base lotion including 0.4% oleic acid, 18:1, was topically administered to a skin area of a subject. The base lotion was substantially free of any retinoid. To administer the base lotion, 0.1 mL of the base lotion was applied to a 0.75 in square bandage, and the bandage was applied to a skin area on the subject's forearm. The base lotion was administered twice per day for five days. No substantial signs of erythema are observed at the treated skin area.

The amount of retinol in the base lotion at the end of administration was measured using HPLC analysis. The amount of retinol in the base lotion at the end of administration was 0%.

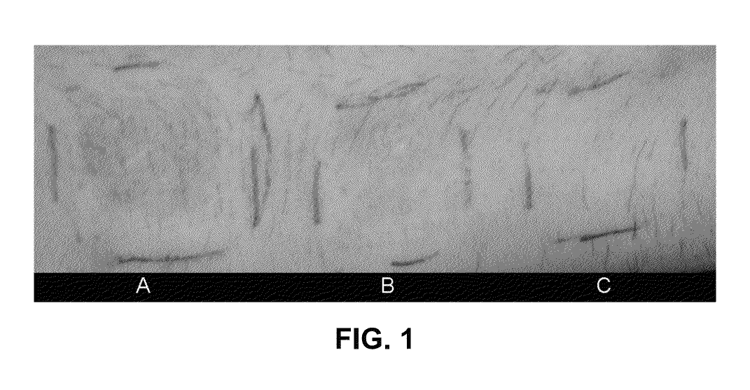

FIG. 1 are color photographs of the treated skin areas from Examples 1-3. Skin area A was treated with the lotion including retinol without a fatty acid (Example 1). Skin area B was treated with the lotion composition including retinol and oleic acid (Example 2). Skin area C was treated with the base lotion including oleic acid without a retinoid (Example 3). The photographs were taken with a flash to better observe differentiation. As shown in FIG. 1, skin area B showed reduced signs of erythema compared to skin area A, demonstrating that the presence of the fatty acid reduced the skin irritation associated with the retinoid therapy.

The amounts of retinol in the lotion of Example 1 and the lotion composition of Example 2 at the end of administration were similar, indicating that the observed reduction in skin irritation was not a result of a loss of retinol activity.

### Example 4

A lotion including 0.4% retinol was topically administered to a skin area of a subject. The lotion was substantially free of any fatty acid. To administer the lotion, 0.1 mL of the lotion was applied to a 0.75 in square bandage, and the bandage was applied to a skin area on the subject's forearm. The lotion was administered once per day for fourteen days, at which time erythema was observed at the treated skin area. The TEWL was measured at the treated skin area. TEWL was measured at each treatment site in triplicate at room temperature and in a humidity-controlled environment using a Vapometer™ (Delfin, Stamford, Conn.).

### Example 5

A base lotion including 1% oleic acid, 18:1, was topically administered to a skin area of a subject. The lotion was substantially free of any retinoid. To administer the base lotion, 0.1 mL of the base lotion was applied to a 0.75 in square bandage, and the bandage was applied to a skin area on the subject's forearm. The base lotion was administered once per day for fourteen days. No substantial signs of erythema were observed at the treated skin area. The TEWL was measured at the treated skin area.

### Example 6

Oleic acid, 18:1, was added to the lotion used in Example 4 to form a lotion composition including 0.4% retinol and 1% oleic acid. The lotion composition was topically administered to a subject. To administer the lotion composition, 0.1 mL of the lotion composition was applied to a 0.75 in square bandage, and the bandage was applied to a skin area on the subject's forearm. The lotion composition was administered once per day for fourteen days, at which time erythema was observed at the treated skin area. The TEWL was measured at the treated skin area.

### Example 7

Oleic acid, 18:1, was added to the lotion used in Example 4 to form a lotion composition including 0.4% retinol and 0.25% oleic acid. The lotion composition was topically administered to a subject. To administer the lotion composition, 0.1 mL of the lotion composition was applied to a 0.75 in square bandage, and the bandage was applied to a skin area on the subject's forearm. The lotion composition was administered once per day for fourteen days, at which time erythema was observed at the treated skin area. The TEWL was measured at the treated skin area.

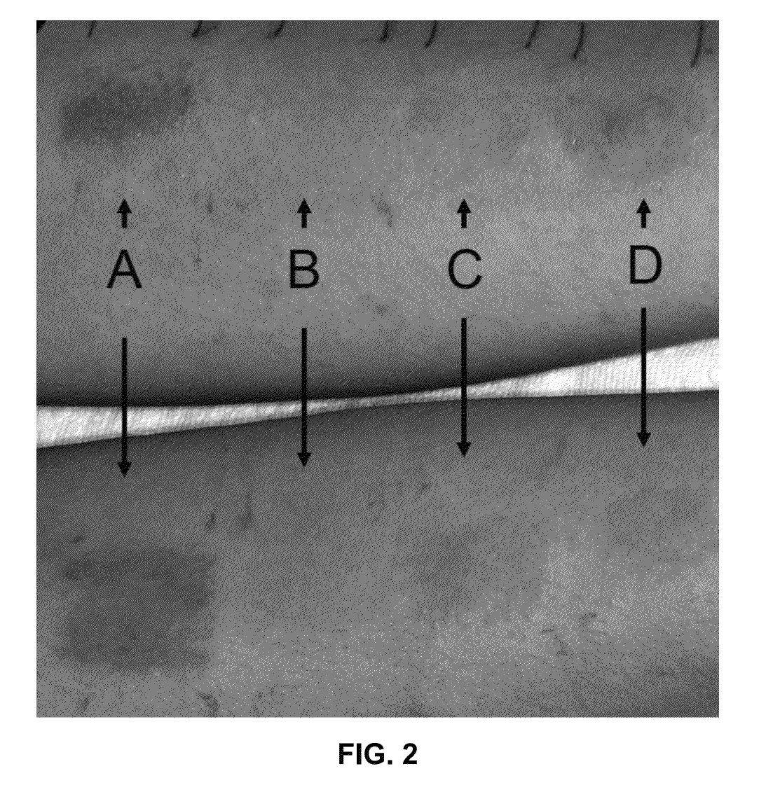

FIG. 2 are color photographs of the treated skin areas from Examples 4-7. Skin area A was treated with the lotion including retinol without a fatty acid (Example 4). Skin area B was treated with the base lotion including oleic acid without a retinoid (Example 5). Skin area C was treated with the lotion composition including 0.4% retinol and 1% oleic acid (Example 6). Skin area D was treated with the lotion composition including 0.4% retinol and 0.25% oleic acid (Example 7). The photographs were taken with a flash to better observe differentiation. As shown in FIG. 2, skin areas C and D showed reduced signs of erythema compared to skin area A, indicating that the presence of the fatty acid reduced the skin irritation associated with the retinoid therapy. Skin area C also showed reduced signs of erythema compared to skin area D, indicating that the reduced skin irritation was dependent on the dosage of the fatty acid (i.e., that the fatty acid reduced retinoid-based inflammation in a dose dependent manner).

Table 1 illustrates a TEWL measurement of the untreated skin area as well as the TEWL measurements from Examples 4-7. A decrease in TEWL is an indicator of improved skin barrier function.

TABLE 1

| | | Oleic | TEWL |
|---|---|---|---|
| Example No. | Retinol (%) | Acid (%) | (g/m² · h) |
| untreated | 0 | 0 | 11.5 |
| 4 | 0.4 | 0 | 302.3 |
| 5 | 0 | 1 | 7.6 |
| 6 | 0.4 | 1 | 27.7 |
| 7 | 0.4 | 0.25 | 177.3 |

As illustrated by Example 4, application of the lotion including the retinoid without the fatty acid increased the TEWL of the treated skin area compared to the untreated skin area (i.e., the retinoid therapy resulted in an increase in TEWL). Application of the lotion of Example 4 increased the TEWL of the treated skin area by 290.8g/m²·h compared to the untreated skin area. Examples 6 and 7 illustrate that addition of the fatty acid to the lotion including the retinoid diminished the increase in the TEWL of the treated skin area. For example, application of the lotion composition of Example 6 increased the TEWL of the treated skin area by 16.2g/m²·h compared to the untreated skin area, which was 274.6g/m²·h less than the increase in TEWL caused by application of the lotion of Example 4. Application of the lotion composition of Example 7 increased the TEWL of the treated skin area by 165.8g/m²·h compared to the untreated skin area, which was 125g/m²·h less than the increase in TEWL caused by application of the lotion of Example 4. The diminished increase in the TEWL achieved with Examples 6 and 7 is indicative of improved skin barrier function resulting from administration of the retinoid with the fatty acid as compared to administration of the retinoid without the fatty acid.

### Example 8

Human kidney cells were stably transfected with a retinoic acid response element (RARE) construct. The transfected cells were exposed to increasing amounts of retinoic acid (from about 0.005 μM to about 10 μM) in the presence of increasing amounts of oleic acid (about 0 μM, about 25 μM, about 50 μM, about 100 μM, and about 200 μM), and the response was measured in relative light units (RLUs).

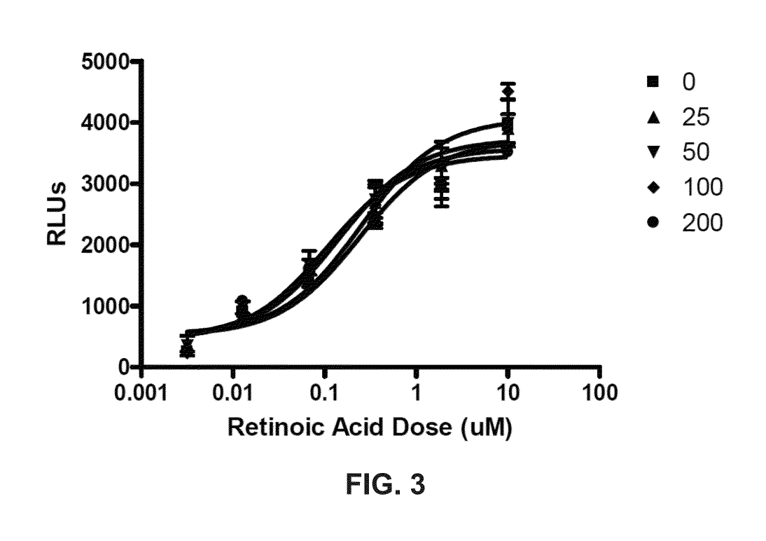

FIG. 3 illustrates the results of Example 8. As shown in FIG. 3, the human kidney cells stably transfected with the RARE construct responded in a dose dependent manner to increasing amounts of retinoic acid in the presence of increasing amounts of oleic acid, which indicates that the presence of the fatty acid did not affect retinoic acid activation of the retinoic acid receptor. This suggests that the fatty acid does not interrupt the retinoic acid signaling to the retinoic acid receptor.

### Example 9

Chinese hamster ovary cells were stably transfected with peroxisome proliferator activated receptor (PPAR) construct and luciferase reporter vector.

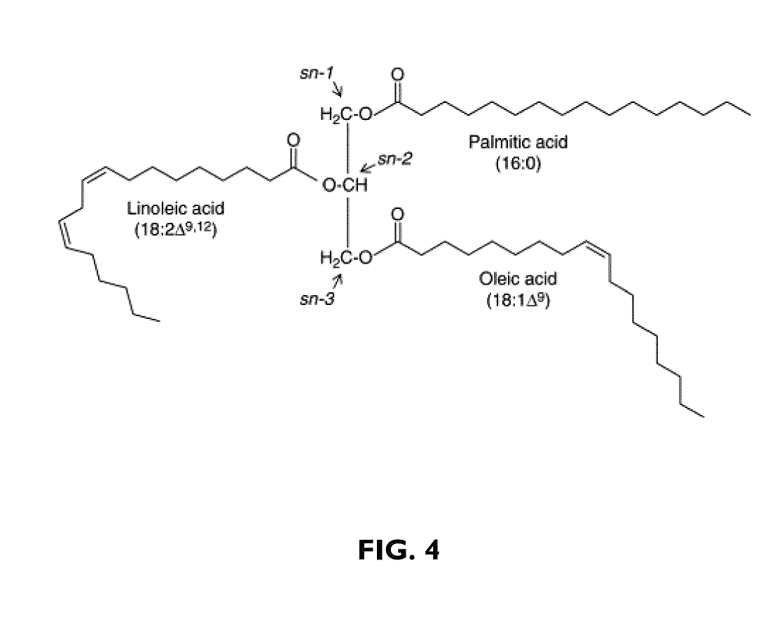

The transfected cells were exposed to increasing amounts of oleic acid 18:1 (about 0 μg/mL, about 6.25 μg/mL, about 12.5 μg/mL, about 25 μg/mL, about 50 μg/mL, and about 100 μg/mL) and increasing amounts of Borage Oil (about 0 μg/mL, about 6.25 μg/mL, about 12.5 μg/mL, about 25 μg/mL, about 50 μg/mL, and about 100 μg/mL) that includes oleic acid as a side chain (as shown in FIG. 4; Borage Oil triglyceride with palmitic, linoleic and oleic acid (18:1) as side chains (Cahoon et al., “Metabolic Engineering of the Content and Fatty Acid Composition of Vegetable Oils,” Pergamon, Oxford, 2008)). The response was measured in relative light units (RLUs).

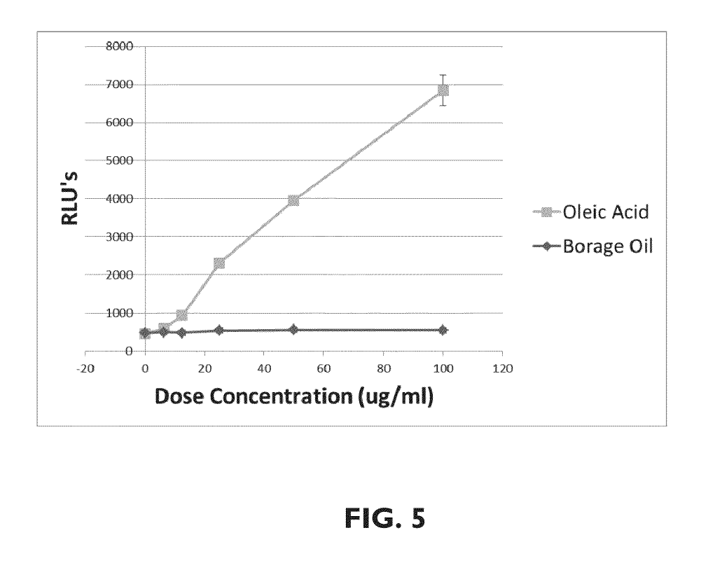

FIG. 5 illustrates the results of Example 9. As shown in FIG. 5, the Chinese hamster cells stably transfected with the PPAR construct and a luciferase reporter vector responded in a dose dependent manner to increasing amounts of oleic acid 18:1, but did not respond to increasing amounts of bound oleic acid found in Borage Oil.

While the present invention has been described with reference to specific exemplary embodiments, it will be evident that various modifications and changes may be made to these embodiments without departing from the spirit and scope of the invention. It is the following claims, including all equivalents, which are intended to define the spirit and scope of the invention.

1. A method for limiting a side effect associated with retinoid therapy comprising topically administering a retinoid to a skin area of a subject; and topically administering a free fatty acid to the skin area of the subject prior to, during, and/or after administration of the retinoid.

2. The method of claim 1, wherein the side effect comprises accelerated barrier disruption and the administering of the free fatty acid limits the accelerated barrier disruption.

3. The method of claim 2, wherein the accelerated barrier disruption is measured by transepidermal water loss (TEWL), and the TEWL at the skin area subsequent to administration of the free fatty acid is less than about 300 g/m²·h.

4. The method of claim 3, wherein the TEWL at the skin area is less than about 200 g/m²·h.

5. The method of claim 2, wherein the accelerated barrier disruption is measured by TEWL, and the TEWL at the skin area following administration of the retinoid and the fatty acid is at least about 40% less compared to the TEWL at the skin area following administration of the retinoid without the fatty acid.

6. The method of claim 5, wherein the TEWL at the skin area following administration of the retinoid and the fatty acid is at least about 90% less compared to the TEWL at the skin area following administration of the retinoid without the fatty acid.

7. The method of claim 1, wherein the retinoid and the fatty acid are administered concurrently to the skin area of the subject.

8. The method of claim 7, wherein the retinoid and the fatty acid are present in a composition in a form suitable for topical administration to the subject.

9. The method of claim 1, wherein the administering of the retinoid is done to treat wrinkles, aging related skin changes, abnormal or diminished synthesis of collagen and elastin, diminished levels of collagen and elastin in the dermis, stretch marks, skin lines, fine lines, thinning of skin, skin thickening due to elastosis of photoaging, loss or reduction of skin resiliency, elasticity and recoilability, lack of skin resiliency, elasticity and recoilability, and older looking skin.

10. The method of claim 1, wherein the retinoid is selected from the group consisting of retinoic acid, all-trans retinoic acid, 13-cis retinoic acid, 9-cis retinoic acid, retinol, retinal, retinoic acid esters, and mixtures thereof.

11. The method of claim 1, wherein the fatty acid is selected from the group of consisting of fatty acids that activate PPAR alpha receptor.

12. The method of claim 1, wherein the side effect comprises at least one of epidermal or dermal irritation.

13. The method of claim 12, wherein the administering of the free fatty acid reduces a visible sign of epidermal or dermal irritation.
